(19) United States
(12) Patent Application Publication
(10) Pub. No.: US 2025/0319000 A1
(43) Pub. Date: Oct. 16, 2025

(54) Oil-in-water emulsion comprising a semiochemical composition, suitable for administration in the form of a cream

(71) Applicant: SIGNS, APT (FR)

(72) Inventors: Maréva REBOUL, APT (FR); Mélinda TEP, APT (FR); Patrick PAGEAT, APT (FR)

(21) Appl. No.: 19/034,757

(22) Filed: Jan. 23, 2025

(30) Foreign Application Priority Data
Apr. 5, 2024 (FR) 2403564

(51) Int. Cl. A61K 8/06; A61K 8/34; A61K 8/37; A61K 8/73; A61Q 19/00

(52) U.S. Cl. CPC A61K 8/062; A61K 8/345; A61K 8/37; A61K 8/73; A61Q 19/00

(57) ABSTRACT

The invention relates to the field of semiochemicals, and describes in particular a composition in the form of an oil-in-water emulsion comprising an aqueous phase, and an oily phase comprising a semiochemical composition, in particular suitable for administration in the form of a cream, as well as its preparation process. The invention also relates to the non-therapeutic use of the composition according to the invention for topical, in particular cutaneous, administration to an animal or human.

### TECHNICAL FIELD OF THE INVENTION

The invention relates to the field of semiochemicals, and describes in particular a composition in the form of an oil-in-water emulsion comprising an aqueous phase, and an oily phase comprising a semiochemical composition, in particular suitable for administration in the form of a cream, as well as its preparation process. The invention also relates to the non-therapeutic use of the composition according to the invention for topical, in particular cutaneous, administration to an animal or human.

### PRIOR STATE OF THE ART

Semiochemicals according to the invention correspond to mixtures of several chemical compounds, described for the first time in particular by Silverstein, R. M. (1977). Semiochemicals are chemical substances released by a plant or animal that provoke a non-therapeutic behavioural or physiological response in another organism. When the semiochemical affects an individual of the same species (“intra-specific semiochemical product”), it is called a pheromone (Wyatt T. D., 2003). There are several types of pheromones that enable different types of behavioural interaction or communication, such as soothing pheromones, sex pheromones, territorial marking pheromones and warning pheromones. When the semiochemical product affects an individual of a different species (“interspecific semiochemical product”), it is called an allelochemical substance. Among allelochemical substances, an allomone is an allelochemical substance that triggers a reaction in the target species, this reaction being favourable to the species that releases the allomone (the opposite is a kairomone).

Since 1995, specialties whose active ingredients are semiochemical agents have been marketed. Some of their uses require the product to be deposited on the animal's skin, for example to obtain the desired effects on an animal in an open environment or to protect it from pests such as parasites or biting insects, or on human skin. The physico-chemical constraints of semiochemical components, and in particular their volatility and insolubility in water, have led to the use of excipients such as ethoxydiglycol in marketed specialties. The use of this type of excipient is now increasingly restricted by current regulations, but also by the general public, tending to reject them because of the way they are synthesised and their potential direct and environmental toxicity. It therefore appeared necessary to develop aqueous formulations of semiochemical compounds.

### DISCLOSURE OF THE INVENTION

The aim of the invention is to develop a composition based on semiochemical products (also referred to as compounds, active ingredients or agents) in a new galenic form, preferably non-toxic and environmentally friendly, in particular in the form of an oil-in-water (O/W) emulsion, preferably in the form of a stable and homogeneous oil-in-water (O/W) emulsion, for topical, in particular cutaneous, administration to an animal, in particular a mammal, or a human, or for topical, in particular cutaneous, administration to farm animals treated as a group, in particular for prolonged release of the semiochemical composition. Preferably, topical administration of the composition according to the invention is carried out by spot or localised deposits at one or more points on the skin, for example by a “spot-on” type deposit, from which the semiochemical composition is released.

### SUMMARY OF THE INVENTION

The invention is defined by the independent claims. The dependent claims define particular or preferred embodiments of the invention.

According to a first aspect, the invention relates to a composition in the form of an oil-in-water (O/W) emulsion (referred to as an O/W composition or composition according to the invention), suitable in particular for the release of semiochemical compounds, comprising:
a) an aqueous phase comprising water, at least one humectant chosen from polyols and sugars, and at least one gelling agent chosen from polysaccharides,
b) an oily phase comprising at least one emollient agent, at least one emulsifying agent, and a semiochemical composition,
   wherein said semiochemical composition comprises:
methyl linoleate, methyl oleate and methyl palmitate; or
bis(2-ethylhexyl) adipate and 1-isopropyl-2,2-dimethyltrimethylene diisobutyrate.

According to a second aspect, the invention relates to a process for preparing a composition according to the invention, comprising the following steps:
a) said at least one gelling agent is pasted into said at least one humectant; then water is added with stirring and at a temperature of between 65° C. and 75° C., preferably at a temperature of 70° C., so as to obtain a homogeneous aqueous phase;
b) said at least one emollient agent, said at least one emulsifying agent and said semiochemical composition and optionally said at least one additive are mixed, with stirring and at a temperature of between 65° C. and 75° C., preferably at a temperature of 70° C., so as to obtain a homogeneous oily phase;
c) the oily phase from step b) is added, with stirring, to the aqueous phase from step a) so as to obtain an oil-in-water (O/W) emulsion;
d) optionally, the pH of the emulsion obtained in step c) is adjusted;
e) optionally, a perfume is added;
f) the composition is recovered in the form of an oil-in-water (O/W) emulsion, particularly stable and homogeneous, obtained in step c), d) or e).

According to a third aspect, the invention relates to the non-therapeutic use of a composition according to the invention for topical, in particular cutaneous, administration to an animal or a human, in particular on the skin of an animal or a human, in particular for sustained release of the semiochemical composition.

### BRIEF DESCRIPTION OF THE FIGURES

FIG. 1 shows the analysis of a compress before the comparative composition was applied to the rabbit's skin.

FIG. 2 shows the analysis of the sample taken 24 hours after the comparative composition was applied to the rabbit's skin.

FIG. 3 shows the analysis of the extract 2 days after the comparative composition was applied to the rabbit's skin.

FIG. 4 shows the analysis of the cotton bud extract before the composition according to the invention was applied to the rabbit's skin.

FIG. 5 shows the analysis of the extract from a cotton bud rubbed 24 hours after the composition according to the invention was applied to the rabbit's skin.

FIG. 6 shows a superposition of the chromatograms obtained for analyses at T=1 day, 4 days, 7 days and 9 days for methyl palmitate (peak between 13.3 and 13.4), methyl linoleate (peak between 16.8 and 16.9) then methyl oleate (peak between 17.0 and 17.1), of the composition according to the invention applied to the rabbit's skin.

### DETAILED DESCRIPTION OF THE INVENTION

The invention is now described in more detail and in a non-limiting way in the following description. Unless otherwise stated, all percentages relating to quantities are percentages by mass. Any reference to ranges of values in the description and/or claims implies, unless otherwise stated, that the limits of the ranges are included.

The invention relates to a composition in the form of an oil-in-water (O/W) emulsion, suitable in particular for the release of semiochemical compounds, comprising:
a) an aqueous phase comprising water, at least one humectant chosen from polyols and sugars, and at least one gelling agent chosen from polysaccharides,
b) an oily phase comprising at least one emollient agent, at least one emulsifying agent, and a semiochemical composition,
   wherein said semiochemical composition comprises:
methyl linoleate, methyl oleate and methyl palmitate; or
bis(2-ethylhexyl) adipate and 1-isopropyl-2,2-dimethyltrimethylene diisobutyrate.

In a particular embodiment of the invention, said semiochemical composition comprises:
methyl linoleate, methyl oleate, methyl palmitate, 2-(2-butoxyethoxy) ethanol, dimethyl glutarate, dimethyl adipate, dimethyl succinate and dodecane; or
methyl stearate, methyl linoleate, methyl palmitate, methyl laurate, methyl myristate and methyl oleate; or
methyl laurate, methyl myristate, methyl palmitate, methyl stearate, methyl oleate, methyl linoleate, dimethyl pimelate and dimethyl azelate; or
methyl linoleate, methyl oleate, methyl palmitate, methyl stearate, methyl myristate, methyl pentadecanoate and methyl laurate; or
bis(2-ethylhexyl) adipate and 1-isopropyl-2,2-dimethyltrimethylene diisobutyrate; or
methyl oleate, methyl palmitate, methyl linoleate, dioxolane, methyl laurate and methyl myristate.

In a preferred embodiment of the invention, said semiochemical composition comprises:
from 30% to 40% by weight of methyl linoleate, from 25% to 35% by weight of methyl oleate, from 15% to 25% by weight of methyl palmitate, from 5% to 15% by weight of 2-(2-butoxyethoxy) ethanol, from 2% to 6% by weight of dimethyl glutarate, from 1% to 3% by weight of dimethyl adipate, from 1% to 3% by weight of dimethyl succinate and from 1% to 3% by weight of dodecane; or
from 5% to 15% by weight of methyl stearate, from 10% to 25% by weight of methyl linoleate, from 15% to 25% by weight of methyl palmitate, from 5% to 15% by weight of methyl laurate, from 5% to 15% by weight of methyl myristate and from 25% to 35% by weight of methyl oleate; or
from 2% to 6% by weight of methyl laurate, from 2% to 6% by weight of methyl myristate, from 11% to 15% by weight of methyl palmitate, from 2% to 6% by weight of methyl stearate, from 42% to 50% by weight of methyl oleate, from 4% to 8% by weight of methyl linoleate, from 7% to 13% by weight of dimethyl pimelate and from 6% to 11% by weight of dimethyl azelate; or
from 25% to 35% by weight of methyl linoleate, from 25% to 35% by weight of methyl oleate, from 15% to 25% by weight of methyl palmitate, from 10% to 15% by weight of methyl stearate, from 2% to 7% by weight of methyl myristate, from 2% to 6% by weight of methyl pentadecanoate and from 1% to 3% by weight of methyl laurate; or
from 40% to 60% by weight of bis(2-ethylhexyl) adipate and from 40% to 60% by weight of 1-isopropyl-2,2-dimethyltrimethylene diisobutyrate;
from 35% to 45% by weight of methyl oleate, from 20% to 30% by weight of methyl palmitate, from 20% to 30% by weight of methyl linoleate, from 2% to 7% by weight of dioxolane, from 1% to 5% by weight of methyl laurate and from 1% to 4% by weight of methyl myristate; the total sum of the compounds present in said semiochemical composition being equal to 100%.

### Galenics

The composition according to the invention is in the form of an oil-in-water (O/W) emulsion, in particular an oil-in-water emulsion which is stable and has a homogeneous appearance, particularly to the naked eye. In particular, the composition is in the form of a cream, preferably in the form of a white cream with a thick, opaque texture.

In the context of the invention, the term “emulsion” refers to a mixture of two immiscible liquid substances, such as oil and water. In an oil-in-water (O/W) emulsion, the oil is dispersed in the water in the form of droplets, particularly small droplets.

In the context of the invention, the term “stable emulsion” refers to an emulsion showing no signs of instability (phase separation, increase in droplet size, etc.), in particular an emulsion which does not release water or oil and which remains in its initial form over time and under different temperature conditions, for example during a 3 months storage at a temperature of between 5° C. and 40° C.

In the context of the invention, the term “homogeneous emulsion” refers to an emulsion whose surface is smooth and uniform to the naked eye.

### Aqueous Phase

The composition according to the invention comprises an aqueous phase, in particular a continuous aqueous phase (also known as the external phase).

The aqueous phase of the composition according to the invention comprises water, preferably demineralised water or purified water, and optionally one or more water-miscible or at least partly water-miscible compounds, for example a skin care agent such as ALOE VERA, a hydrolat, urea or an antistatic agent.

In the context of the invention, the term “skin care agent” refers to an agent for maintaining the skin in good condition, e.g. healthy-looking.

In the context of the invention, the term “antistatic agent” refers to an agent that reduces static electricity by neutralising the electrical charge on a surface.

In a particular embodiment of the invention, the composition according to the invention comprises from 60% to 95% by weight of water, preferably from 70% to 80% by weight of water, relative to the total weight of the composition.

In a preferred embodiment of the invention, the composition according to the invention comprises from 62% to 91% by weight of water, preferably from 73.7% to 76% by weight of water, even more preferably 77.9% by weight of water, relative to the total weight of the composition.

The aqueous phase of the composition according to the invention may comprise water, at least one humectant chosen from polyols and sugars, and at least one gelling agent chosen from polysaccharides.

### Humectant

According to a particular embodiment, the aqueous phase of the composition according to the invention also comprises at least one humectant chosen from polyols and sugars. In particular, the aqueous phase of the composition according to the invention also comprises a single humectant chosen from polyols or sugars, preferably polyols. Said at least one humectant is notably soluble in the aqueous phase.

In the context of the invention, the term “humectant” (also called moisturising agent) refers to a compound or mixture of compounds that retain water thanks to their hygroscopic properties.

In the context of the invention, the term “polyol” refers to any organic compound comprising at least two free hydroxyl groups (—OH), such as glycols.

In the context of the invention, the term “sugar” refers to simple carbohydrates, in particular any organic molecule consisting of one ose (monosaccharide) or two oses (disaccharide).

In a particular embodiment of the invention, said at least one humectant is chosen from polyols selected from the group consisting of glycerol (also called glycerin), propylene glycol, polyethylene glycols (PEGs), xylitol, isomalt, maltitol, mannitol, polydextrose, sorbitol, isomaltitol, lactitol, and mixtures thereof; and/or said at least one humectant is chosen from sugars selected from the group consisting of fructose, glucose, lactose and mixtures thereof.

In a preferred embodiment of the invention, said at least one humectant is a polyol, in particular glycerol.

Said at least one humectant, i.e. one or more humectants, preferably a humectant, may be present in the composition in an amount ranging from 4% to 12% or 4.5% to 12% by weight, preferably from 7% to 8% by weight, relative to the total weight of the composition.

Preferably, said at least one humectant is glycerol and is present in the composition in an amount ranging from 4.5% to 12% by weight, preferably 7.5% by weight, relative to the total weight of the composition.

### Gelling Agent

In a particular embodiment, the aqueous phase of the composition according to the invention also comprises at least one gelling agent chosen from polysaccharides. In particular, the aqueous phase of the composition according to the invention also comprises a single gelling agent chosen from polysaccharides. Said at least one gelling agent is notably soluble in the aqueous phase. Said at least one gelling agent is a hydrophilic gelling agent.

In the context of the invention, the term “gelling agent” refers to an agent capable of increasing the viscosity of the oil-in-water emulsion and/or allowing the various constituents of the composition to adhere together.

In the context of the invention, the term “polysaccharides” (also known as glycans, polyosides or polyholosides) refers to complex carbohydrates formed by the polymerisation of oses (monosaccharides) linked together by glycosidic bonds.

In a particular embodiment of the invention, the polysaccharides are selected from the group consisting of xanthan gum, guar gum, cellulose, cellulose derivatives, maltodextrin, maltodextrin derivatives and mixtures thereof, preferably xanthan gum.

The polysaccharides may be present in the composition in an amount ranging from 0.05% to 1% or 0.05% to 0.9% by weight, preferably from 0.1% to 0.6% by weight, relative to the total weight of the composition.

Preferably, the polysaccharides are xanthan gum and are present in the composition in an amount ranging from 0.05% to 0.9% by weight, preferably from 0.2% to 0.5% by weight, even more preferably 0.5% by weight, relative to the total weight of the composition.

In a particular embodiment of the invention, the aqueous phase of the composition comprises glycerol in an amount ranging from 4.5% to 12% by weight, preferably 7.5% by weight, and xanthan gum in an amount ranging from 0.05% to 0.9% by weight, preferably from 0.2% to 0.5% by weight, even more preferably 0.5% by weight, all the percentages being given relative to the total weight of the composition.

### Oily Phase

The composition according to the invention comprises an oily phase, in particular a dispersed oily phase (also called discontinuous or internal phase).

The oily phase of the composition according to the invention may comprise at least one emollient agent, at least one emulsifying agent and a semiochemical composition. In particular, said at least one emollient agent, said at least one emulsifying agent and said semiochemical composition are notably soluble in the oily phase.

The oily phase may be present in an amount ranging from 3% to 30% by weight, preferably 15% to 25% by weight, relative to the total weight of the composition.

### Emollient Agent

According to a particular embodiment, the oily phase of the composition according to the invention also comprises at least one emollient agent. In particular, the oily phase of the composition according to the invention also comprises a single emollient agent. Said at least one emollient agent is notably soluble in the oily phase.

In the context of the invention, the term “emollient agent” refers to an agent to soften the skin and increase its suppleness during topical, in particular cutaneous, administration of the composition according to the invention.

In a particular embodiment of the invention, said at least one emollient agent is selected from the group consisting of hydrogenated or non-hydrogenated vegetable oils (vegetable oils comprising triglycerides of unsaturated fatty acids, for example olive oil, rapeseed oil, sunflower oil, sesame oil, linseed oil, hemp oil, walnut oil, hazelnut oil, coconut oil, argan oil, jojoba oil, sweet almond oil, shea butter, castor oil, avocado oil, apricot kernel oil, macadamia oil, . . . ), silicone oils (e.g., dimethicone, . . . ), mineral oils and waxes (e.g., Parrafine, . . . ), fatty alcohols (e.g., arachidyl alcohol, batyl alcohol, behenyl alcohol, C₁₂-C₁₃ alcohols, cetearyl alcohol, cetyl alcohol, coconut alcohol, isocetyl alcohol, octyldodecanol, palm alcohol, . . . ), fatty acids and fatty acid esters (e.g., batyl stearate, cetearyl behenate, cetearyl isononanoate, cetyl esters, coconut acid, ethyl linoleate, ethyl linolenate, ethyl olivate, ethylhexyl cocoate, ethylhexyl myristate, glyceryl caprylate, glyceryl laurate/oleate, glyceryl ricinoleate, isocetyl salicylate, isostearyl alcohol, linoleic acid, linolenic acid, oleic acid, palmitic acid, palmitic acid, wheat germ acid, . . . ), glyceride derivatives (e.g., triglycerides of capric/caprylic acid, cocoglycerides, palm glycerides, . . . ) and glycerol derivatives (e.g., glycerol trioctanoate: Tricaprylin, glycerol tristearate: Tristearin . . . ), preferably glyceride derivatives such as fatty acid triglycerides.

Said at least one emollient agent, i.e. one or more emollient agent(s), is/are present in an amount ranging from 0.5% to 8% by weight, preferably from 1% to 3% by weight, relative to the total weight of the composition.

Preferably, said at least one emollient agent is a glyceride derivative, in particular a fatty acid triglyceride, in particular a caprylic/capric acid triglyceride such as that marketed in particular by BASF under the name Myritol 318®, and is present in the composition in an amount ranging from 0.5% to 8% by weight, preferably 2% by weight, relative to the total weight of the composition.

In a particular embodiment of the invention, the aqueous phase of the composition comprises glycerol in an amount ranging from 4.5% to 12% by weight, preferably 7.5% by weight, and xanthan gum in an amount ranging from 0.05% to 0.9% by weight, preferably from 0.2% to 0.5% by weight, even more preferably 0.5% by weight, and the oily phase of the composition comprises a caprylic/capric acid triglyceride such as Myritol 318® in an amount ranging from 0.5% to 8% by weight, preferably 2% by weight, all the percentages being given relative to the total weight of the composition.

### Emulsifying Agent

The oily phase of the composition according to the invention also comprises at least one emulsifying agent, preferably one or more emulsifying agent(s). Said at least one emulsifying agent is notably soluble in the oily phase.

In the context of the invention, the term “emulsifying agent” (also called emulsifier) refers to a small molecule composed of a hydrophilic head soluble in the aqueous phase and a lipophilic tail with an affinity for the oily phase. Said at least one emulsifying agent enables the formation of an interfacial film around the droplets in the dispersed phase, thus forming the emulsion and providing stability to the formed emulsion.

Said at least one emulsifying agent may be chosen from non-ionic or ionic (anionic, cationic) surfactants, preferably non-ionic surfactants.

In the context of the invention, the term “surfactant” refers to a compound which reduces surface tension and promotes uniform distribution of the composition according to the invention when used.

In a particular embodiment of the invention, said at least one non-ionic surfactant has a hydrophilic-lipophilic balance (HLB) of between 8 and 20 at 25° C.

In a particular embodiment of the invention, said at least one emulsifying agent is an ionic surfactant, preferably an anionic surfactant chosen from sulphated castor oil, sodium dodecyl sulphate (SDS), sodium coco sulphate (INCI name: Sodium coco sulfate (SCS)), sodium cocoyl isethionate (INCI name: Sodium cocoyl isethionate (SCI)), sodium lauryl sulphoacetate (INCI name: Sodium lauryl sulfoacetate (SLSA)), sodium laureth sulphate (INCI name: Sodium laureth sulfate (SLES)) and sodium lauroyl sarcosinate (INCI name: Sodium Lauroyl Sarcosinate), or a cationic surfactant chosen from cetearyl alcohol (INCI name: Cetearyl Alcohol), behentrimonium methosulphate (INCI name: Behentrimonium Methosulfate (BTMS)), brassica alcohol (and) brassicycle valinate esylate (INCI name: Brassica Alcohol (and) Brassicyl Valinate Esylate), benzalkonium chloride (BAC), didecyldimethylammonium chloride (DDAC), brassicamidopropyl dimethylamine (INCI name: Brassicamidopropyl dimethylamine), Polyquaternium-7, Polyquaternium-10, Polyquaternium-22 and Polyquaternium-37.

In a preferred embodiment of the invention, said at least one emulsifying agent is a non-ionic surfactant, preferably a non-ionic surfactant having an HLB of between 8 and 20 at 25° C., and is chosen in particular from polyoxyethylene sorbitan fatty acid esters, alkyl polyglucosides, or mixtures of at least one alkyl polyglucoside and at least one fatty alcohol.

Examples of polyoxyethylene sorbitan fatty acid esters (also called esters of fatty acid and polyoxyethylene sorbitan; or ethoxylated sorbitan, sorbitan ester) include in particular esters of C₈-C₂₄, preferably C₁₆-C₂₂, fatty acids and of polyoxyethylene sorbitan which may comprise from 1 to 150 oxyethylenated groups, such as polysorbate 20 in particular marketed under the name Tween 200 by CRODA, polysorbate 21 in particular marketed under the name Tween 21® by CRODA, polysorbate 40 in particular marketed under the name Tween 40® by CRODA, polysorbate 60 in particular marketed under the name Tween 60® by CRODA, polysorbate 61 in particular marketed under the name Tween 61® by CRODA, polysorbate 65 in particular marketed under the name Tween 65® by CRODA, polysorbate 80 in particular marketed under the name Tween 80® by CRODA, polysorbate 81 in particular marketed under the name Tween 81® by CRODA and polysorbate 8, in particular marketed under the name Tween 85® by CRODA, preferably polysorbate 80 and polysorbate 85.

Examples of alkyl polyglucosides (APGs) are known to those skilled in the art.

Mixtures of at least one alkyl polyglucoside and at least one fatty alcohol include in particular at least one C₁₂-C₂₀ alkyl polyglucoside and at least one C₁₄-C₂₂ fatty alcohol such as Montanov L® in particular marketed by the company SEPPIC, a mixture of myristic alcohol and myristylic glucoside such as Montanov 14® in particular marketed by the company SEPPIC.

In a particular embodiment of the invention, said at least one emulsifying agent, i.e. one or more emulsifying agent(s), is/are present in the composition in an amount ranging from 2% to 30% by weight, preferably from 10% to 12% by weight, relative to the total weight of the composition.

In a preferred embodiment of the invention, the composition according to the invention comprises several emulsifying agents, in particular a mixture of emulsifying agents selected from the group consisting of polyoxyethylene sorbitan fatty acid esters, alkyl polyglucosides, and mixtures of at least one alkyl polyglucoside and at least one fatty alcohol, preferably a mixture of emulsifying agents selected from the group consisting of polyoxyethylene sorbitan fatty acid esters, and mixtures of at least one alkyl polyglucoside and at least one fatty alcohol. Preferably, said emulsifying agents are a C₁₂-C₂₀ alkyl polyglucoside and at least one C₁₄-C₂₂ fatty alcohol such as Montanov LR marketed in particular by the company SEPPIC, a mixture of myristic alcohol and myristylic glucoside such as Montanov 14® marketed in particular by the company SEPPIC, polyoxyethylene sorbitan fatty acid esters such as polysorbate 80, in particular marketed under the name Tween 80® by CRODA, and polysorbate 85, in particular marketed under the name Tween 85® by CRODA, and said emulsifying agents are present in the composition in an amount ranging from 2.5% to 28.1% by weight, preferably 11% by weight, relative to the total weight of the composition. In particular, Montanov LR is present in the composition in an amount ranging from 1.3% to 8.5% by weight, preferably 5% by weight, relative to the total weight of the composition; and/or Montanov14® is present in the composition in an amount ranging from 0.2% to 5.2% by weight, preferably 2% by weight, relative to the total weight of the composition; and/or polysorbate 80 is present in the composition in an amount ranging from 0.5% to 7.2% by weight, preferably 2% by weight, relative to the total weight of the composition; and/or polysorbate 85 is present in the composition in an amount ranging from 0.5% to 7.2% by weight, preferably 2% by weight, relative to the total weight of the composition.

In a particular embodiment of the invention, the aqueous phase of the composition comprises glycerol in an amount ranging from 4.5% to 12% by weight, preferably 7.5% by weight, and xanthan gum in an amount ranging from 0.05% to 0.9% by weight, preferably 0.2% to 0.5% by weight, even more preferably 0.5% by weight, and the oily phase of the composition comprises a caprylic/capric acid triglyceride such as Myritol 318® in an amount ranging from 0.5% to 8% by weight, preferably 2% by weight, and a mixture of emulsifying agents consisting of Montanov L® present in the composition in an amount ranging from 1.3% to 8.5% by weight, preferably 5% by weight, Montanov 14® in an amount ranging from 0.2% to 5.2% by weight, preferably 2% by weight, polysorbate 80 in an amount ranging from 0.5% to 7.2% by weight, preferably 2% by weight, and polysorbate 85 in an amount ranging from 0.5% to 7.2% by weight, preferably 2% by weight, all the percentages being given relative to the total weight of the composition.

### Semiochemical Composition

The oily phase of the composition according to the invention also comprises a semiochemical composition as active agent, as defined above. Said semiochemical composition is notably soluble in the oily phase described herein. Particular semiochemical compositions are described above and in the preferred embodiments and experimental examples that follow.

The semiochemical compositions contained in the compositions according to the invention are in particular intended for non-therapeutic use, for example use for their behavioural or protective effect against pests (parasites, insects, etc.) on the animals to which they are administered or on animals interacting with them, in the form of the O/W composition.

The semiochemical composition may be present in the composition in the form of an oil-in-water emulsion in an amount ranging from 2% to 5% by weight, preferably from 2.2% to 4.2% by weight of said semiochemical composition, relative to the total weight of the composition according to the invention.

### Additives

The composition according to the invention may further comprise at least one additive provided that it does not interfere with the desired properties of the composition, in particular with its property of being an O/W emulsion.

In a particular embodiment of the invention, said at least one additive is selected from the group consisting of a preservative, in particular an antimicrobial agent or an antioxidant agent, a perfume, a masking agent, and mixtures thereof.

In the context of the invention, the term “preservative” refers to an agent that protects the composition according to the invention from changes in its physical or chemical state, its colour or its odour, or protects it from microbial contamination or degradation, for example by inhibiting the development of micro-organisms during the time the composition according to the invention is stored or used. Such an agent must be compatible with use of the composition according to the invention in contact with an animal or a human, and is advantageously non-irritating to the skin and eyes.

Examples of preservatives are known to the skilled in the art and include in particular benzyl alcohol, didecyldimonium chloride, caprylyl glycol, phenylpropanol, sodium benzoate, phenyl acetate, sorbic acid, dehydroacetic acid, potassium sorbate, phenoxyethanol, cetrimonium bromide, triclosan, parabens and thiazolinones.

In the context of the invention, the term “antimicrobial agent” therefore refers to a preservative that slows down the growth of micro-organisms (bacteria, fungi, yeasts, moulds) and combats the development of microbes in the composition according to the invention, when it is stored and/or when it is used.

Examples of antimicrobial agents are known to those skilled in the art and include in particular glycerin/phenyl alcohol/caprylhydroxamic acid, marketed in particular under the name Aromastat® by INOLEX.

The composition according to the invention may comprise from 0% to 4% by weight, preferably from 0.2% to 3.2% by weight, even more preferably 1% by weight of preservative, in particular antimicrobial agent, relative to the total weight of the composition according to the invention.

In the context of the invention, the term “antioxidant agent” refers to a preservative capable of inhibiting reactions promoted by oxygen, thereby preventing oxidation.

Examples of antioxidant agents are known to those skilled in the art and include in particular compounds of the cinnamic acid family and their derivatives, such as pentaerythrityl tetra-di-t-butyl hydroxyhydrocinnamate in particular marketed under the name Tinogard TT® by BASF, vitamin A (retinol), vitamin C (ascorbic acid) and derivatives (ascorbate), vitamin E (tocopherol) and derivatives (tocopheryl), preferably Tinogard TT®.

The composition according to the invention may comprise from 0% to 2% by weight, preferably from 0.02% to 0.3% by weight, even more preferably 0.1% by weight of antioxidant agent, relative to the total weight of the composition.

In the context of the invention, the term “perfume” or perfuming agent refers to any hydrophobic substance designating a pure substance or a mixture that is insoluble or sparingly soluble in water and capable of giving off a pleasant odour.

The composition according to the invention may comprise from 0% to 3% by weight, preferably from 0.2% to 2.5% by weight, even more preferably 1% or 2% by weight of perfume, relative to the total weight of the composition.

In the context of the invention, the term “masking agent” refers to an agent that reduces or inhibits the basic odour or taste of the product.

Examples of masking agents are known to the skilled in the art and include in particular beta sitosterol, citric acid, bisabolol and Calendula officinalis flower extract.

The composition according to the invention can comprise from 0% to 3% by weight, preferably from 0.2% to 2.5% by weight, even more preferably 1% or 2% by weight of a masking agent, relative to the total weight of the composition.

In a particular embodiment of the invention, said at least one additive is added to the oily phase, i.e. the oily phase further comprises at least one additive as defined above.

In another particular embodiment of the invention, said at least one additive, for example a perfume, is added after the formation of the oil-in-water emulsion, or after a step for adjusting the pH of the oil-in-water emulsion.

In a preferred embodiment of the invention, the oily phase also comprises at least one additive selected from the group consisting of a preservative, in particular an antimicrobial agent or an antioxidant agent, a perfume, a masking agent, and mixtures thereof, even more preferably at least one additive selected from the group consisting of a preservative, in particular an antimicrobial agent or an antioxidant agent, a masking agent, and mixtures thereof.

In another preferred embodiment of the invention, the composition according to the invention also comprises at least one additive such as a perfume. In particular, said perfume is added after the formation of the oil-in-water emulsion or after a step for adjusting the pH of the oil-in-water emulsion in order to avoid degradation of the perfume or loss of the perfume odour.

In a particular embodiment of the invention, the composition comprises from 60% to 95% by weight of water, preferably from 70% to 80% by weight of water, relative to the total weight of the composition, and/or comprises from 2% to 5% by weight, preferably from 2.2% to 4.2% by weight of said semiochemical composition, relative to the total weight of the composition.

In a preferred embodiment of the invention, the composition comprises:
a) an aqueous phase comprising water, glycerol and xanthan gum,
b) an oily phase comprising at least one C₁₂-C₂₀ alkyl polyglucoside and at least one C₁₄-C₂₂ fatty alcohol (Montanov LR in particular marketed by the company SEPPIC), a mixture of myristic alcohol and myristylic glucoside (Montanov 14® in particular marketed by the company SEPPIC), caprylic/capric acid triglycerides (Myritol 318® in particular marketed by BASF), polyoxyethylene sorbitan fatty acid esters (polysorbate 80 in particular marketed under the name Tween 80® by CRODA and polysorbate 85 in particular marketed under the name Tween 85® by CRODA), pentaerythrityl tetra-di-t-butyl hydroxyhydrocinnamate (Tinogard TT® in particular marketed by BASF), glycerin/phenyl alcohol/caprylhydroxamic acid (Aromastat® in particular marketed by INOLEX) and a semiochemical composition,
the semiochemical composition being present in the composition in the form of an oil-in-water emulsion according to the invention in an amount ranging from 2% to 5% by weight, preferably from 2.2% to 4.2% by weight, relative to the total weight of the composition according to the invention.

In an even more preferred embodiment of the invention, the composition according to the invention comprises: a) an aqueous phase comprising water in an amount ranging from 62% to 91% by weight of water, preferably from 73.7% to 76% by weight of water, even more preferably 77.9% by weight, glycerol in an amount ranging from 4.5% to 12% by weight, preferably 7.5% by weight, and xanthan gum in an amount ranging from 0.05% to 0.9% by weight, preferably from 0.2% to 0.5% by weight of water, even more preferably 0.5% by weight, b) an oily phase comprising at least one C₁₂-C₂₀ alkyl polyglucoside and at least one C₁₄-C₂₂ fatty alcohol (Montanov L®, marketed in particular by SEPPIC) in an amount ranging from 1.3% to 8.5% by weight, preferably 5% by weight; a mixture of myristic alcohol and myristylic glucoside (Montanov 14®, in particular marketed by SEPPIC) in an amount ranging from 0.2% to 5.2% by weight, preferably 2% by weight; triglycerides of caprylic/capric acids (Myritol 318®, in particular marketed by BASF) in an amount ranging from 0.5% to 8% by weight, preferably 2% by weight; polyoxyethylene sorbitan fatty acid esters such as polysorbate 80, in particular marketed under the name Tween 80® by CRODA, in an amount ranging from 0.5% to 7.2% by weight, preferably 2% by weight, and polysorbate 85, in particular marketed under the name Tween 85® by CRODA, in an amount ranging from 0.5% to 7.2% by weight, preferably 2% by weight; pentaerythrityl tetra-di-t-butyl hydroxyhydrocinnamate (Tinogard TT® in particular marketed by BASF) in an amount ranging from 0.02% to 0.3% by weight, preferably 0.1% by weight, glycerin/phenyl alcohol/caprylhydroxamic acid (Aromastat® in particular marketed by INOLEX) in an amount ranging from 0.2% to 3.2% by weight, preferably 1% by weight, and a semiochemical composition in an amount ranging from 2% to 5% by weight, preferably from 2.2% to 4.2% by weight, the amounts being expressed by weight relative to the total weight of the O/W composition according to the invention.

In a particular embodiment of the invention, the composition has a Brookfield viscosity greater than or equal to 4500 mPa·s, in particular between 4500 mPa·s and 5500 mPa·s, preferably 4500 mPa·s, as measured at 25° C. The viscosity within the meaning of the present invention is a Brookfield viscosity measured at room temperature (25° C.), preferably after 24 hours, by means of a viscometer, for example a Brookfield digital viscometer (model LDVI Prime), mobile S 63 and rotation speed 6 RPM.

In a particular embodiment of the invention, the composition in the form of an oil-in-water (O/W) emulsion has a pH of between 5 and 8, preferably a pH of between 5.5 and 7.5 or a pH of between 7 and 8.

Preferably, the composition has a pH value compatible with topical, in particular cutaneous, application of the composition to animal or human skin.

### Preparation Processes for the Composition of the Invention

The invention also relates to a first process for preparing a composition according to the invention, comprising the following steps:
a) said at least one gelling agent is pasted into said at least one humectant; then water is added with stirring and at a temperature of between 65° C. and 75° C., preferably at a temperature of 70° C., so as to obtain a homogeneous aqueous phase;
b) said at least one emollient agent, said at least one emulsifying agent and said semiochemical composition and optionally said at least one additive are mixed, with stirring and at a temperature of between 65° C. and 75° C., preferably at a temperature of 70° C., so as to obtain a homogeneous oily phase;
c) the oily phase from step b) is added, with stirring, to the aqueous phase from step a) so as to obtain an oil-in-water (O/W) emulsion;
d) optionally, the pH of the emulsion obtained in step c) is adjusted;
e) optionally, a perfume is added;
f) the composition is recovered in the form of an oil-in-water (O/W) emulsion obtained in step c), d) or e).

The invention also relates to a second process for preparing a composition according to the invention, comprising the following steps:
a) said at least one gelling agent is pasted into said at least one humectant; then water is added with stirring and at a temperature of between 65° C. and 75° C., preferably at a temperature of 70° C., so as to obtain a homogeneous aqueous phase;
b) said at least one emollient agent, said at least one emulsifying agent and said semiochemical composition are mixed, with stirring and at a temperature of between 65° C. and 75° C., preferably at a temperature of 70° C., so as to obtain a homogeneous oily phase;
c) the oily phase from step b) is added, with stirring, to the aqueous phase from step a) so as to obtain an oil-in-water (O/W) emulsion;
d) optionally, the pH of the emulsion obtained in step c) is adjusted;
e) optionally, said at least one additive is added;
f) the composition is recovered in the form of an oil-in-water (O/W) emulsion obtained in step c), d) or e).

Preferably, the invention relates to the first process for preparing a composition according to the invention.

The definitions and/or particular/preferred embodiments mentioned in the following paragraphs are applicable to both the first and second processes for preparing a composition according to the invention.

In the context of the invention, the term “pasting” means that before adding the water in step a) of the process, the at least one gelling agent is dispersed in the at least one humectant, in order to obtain a smooth and homogeneous paste. This pasting (or mashing) step, known to those skilled in the art, separates the particles and prevents the formation of lumps.

In a particular embodiment of the first or second process according to the invention, the pasting step a) is carried out at room temperature; and/or
the homogeneous aqueous phase obtained in step a) is kept under stirring at a temperature of between 65° C. and 75° C., preferably at a temperature of 70° C.; and/or
the homogeneous oily phase obtained in step b) is kept under stirring at a temperature of between 65° C. and 75° C., preferably at a temperature of 70° C.; and/or
step c) is carried out by mixing the oily phase from step b) in the aqueous phase from step a) at a temperature of between 65° C. and 75° C., preferably at a temperature of 70° C., and the emulsion is cooled to room temperature; and/or
step d) is carried out by adding a basic solution, for example a NaOH solution, in particular a 1% NaOH solution, in order to increase the pH of the emulsion obtained in step c), preferably in order to obtain a pH of between 5 and 8, preferably a pH of between 7 and 8.

In a particular embodiment of the process according to the invention, the aqueous phase obtained in step a) is in the form of a homogeneous mixture.

In a particular embodiment of the process according to the invention, the oily phase obtained in step b) is in the form of a homogeneous mixture.

In the context of the invention, the term “homogeneous mixture” refers to a mixture in which the individual constituents cannot be distinguished by the naked eye.

In a particular embodiment of the process according to the invention, the oil-in-water (O/W) emulsion obtained in step c) is a stable and homogeneous emulsion as defined above.

In a particular embodiment of the process according to the invention, step d) is obligatory for the preparation of a composition in the form of an oil-in-water (O/W) emulsion, comprising a semiochemical composition comprising (i) from 5% to 15% by weight of methyl stearate, from 10% to 25% by weight of methyl linoleate, from 15% to 25% by weight of methyl palmitate, from 5% to 15% by weight of methyl laurate, from 5% to 15% by weight of methyl myristate and from 25% to 35% by weight of methyl oleate; or (ii) from 2% to 6% by weight of methyl laurate, from 2% to 6% by weight of methyl myristate, from 11% to 15% by weight of methyl palmitate, from 2% to 6% by weight of methyl stearate, from 42% to 50% by weight of methyl oleate, from 4% to 8% by weight of methyl linoleate, from 7% to 13% by weight of dimethyl pimelate and from 6% to 11% by weight of dimethyl azelate; preferably (i) 11% by weight of methyl stearate, 17% by weight of methyl linoleate, 22% by weight of methyl palmitate, 11% by weight of methyl laurate, 11% by weight of methyl myristate and 28% by weight of methyl oleate (SC2); or (ii) 4.50% by weight methyl laurate, 4.50% by weight methyl myristate, 13.40% by weight methyl palmitate, 4.50% by weight methyl stearate, 46.60% by weight methyl oleate, 6.50% by weight methyl linoleate, 11% by weight dimethyl pimelate and 9% by weight dimethyl azelate (SC3).

The invention relates in particular to a process for preparing a composition according to the invention, in particular a first or second process for preparing a composition according to the invention as described above, in which the compounds of the aqueous and oily phases are as described above.

The invention also relates to the use of a composition consisting of an aqueous phase and a composition consisting of an oily phase as described above and exemplified below, to produce a composition according to the invention comprising, as active agent, a semiochemical composition (associated with the oily phase), in the form of an oil-in-water (O/W) emulsion, in particular an oil-in-water emulsion which is stable and has a homogeneous appearance. In particular, the composition is in the form of a cream, preferably in the form of a white cream, with an opaque and thick texture (not flowing naturally out of its container).

### Non-Therapeutic Use According to the Invention

According to another aspect, the invention relates to a non-therapeutic use of a composition according to the invention. According to this use, the composition according to the invention is used to obtain the semiochemical effect(s) expected from the semiochemical composition it contains. Thus, a composition according to the invention is used to improve the management of the behaviour or interactions of target animals or even their well-being, by transmitting information or signals vectored by semiochemical agents.

In particular, the invention relates to a non-therapeutic use of a composition in the form of an oil-in-water (O/W) emulsion according to the invention, for topical, in particular cutaneous, preferably localised, administration to an animal, in particular a mammal, or to a human, in particular for sustained release of the semiochemical composition.

One use of the composition according to the invention is, for example, as a tool or means of improving animal husbandry. One use of the composition according to the invention is, for example, as an adaptive tool or means for the animal in relation to its environment.

The use of the composition according to the invention comprises the topical, in particular cutaneous, preferably localised (“spot-on” type) administration of said composition to an animal, in particular a mammal, or to a human, or to animals treated as a group, in particular for prolonged release of the semiochemical composition.

In a particular embodiment of the invention, the composition is administered topically, in particular cutaneously, to the skin of an animal or to human skin, preferably to the skin of an animal. In particular, the composition is administered locally to an animal or human's skin in a single spot (“spot-on”), or to a small area of an animal or human's skin as a straight line along the back, for example along the animal's dorsal line from neck to tail (“line-on”), or as a narrow band along the back, for example along the animal's dorsal line (“pour-on”). Preferably, the composition is administered by “spot-on”, “line-on” or “pour-on” application.

In the context of the invention, the term “sustained release” means that the semiochemical composition according to the invention is released from its galenic form in a progressive, controlled manner over a more or less extended period of time, as opposed to immediate release where the duration of action is shorter.

In a preferred embodiment of the invention, the composition has a persistence time of between 5 and 9 days, thus allowing prolonged release of the semiochemical composition and offering a rate of administration of the composition that is acceptable to users.

In a particular embodiment according to the invention, the animal is a domestic animal or a farm animal, in particular a mammal, the animal being preferably chosen from the group consisting of pets such as rabbits, cats, dogs, birds or from the group of farm animals such as horses or production animals such as pigs, cattle, poultry, for example hens.

In a particular embodiment according to the invention, the composition comprises a semiochemical composition, in particular a soothing rabbit pheromone, comprising from 30% to 40% by weight of methyl linoleate, from 25% to 35% by weight of methyl oleate, from 15% to 25% by weight of methyl palmitate, from 5% to 15% by weight of 2-(2-butoxyethoxy) ethanol, from 2% to 6% by weight of dimethyl glutarate, from 1% to 3% by weight of dimethyl adipate, from 1% to 3% by weight of dimethyl succinate and from 1% to 3% by weight of dodecane; and relates to the non-therapeutic use of said composition for topical, in particular cutaneous, administration to rabbits, in particular on the skin of a rabbit, in particular to promote well-being in the rabbit, for example to promote positive emotional states in the rabbit, in particular to facilitate adaptation to transport, during examination by the vet, during encounters with unknown animals or people, during arrival in an unknown place, or any other circumstance in the daily life of a rabbit.

In a particular embodiment according to the invention, the composition comprises a semiochemical composition, in particular a cat-soothing pheromone, comprising from 5% to 15% by weight of methyl stearate, from 10% to 25% by weight of methyl linoleate, from 15% to 25% by weight of methyl palmitate, from 5% to 15% by weight of methyl laurate, from 5% to 15% by weight of methyl myristate and from 25% to 35% by weight of methyl oleate; and relates to the non-therapeutic use of said composition for topical, in particular cutaneous, administration to cats, in particular on the skin of a cat, in particular to prevent social stress in cats and/or aggressive behaviour induced by encounters with unknown congeners, unknown people or animals of other species.

In a particular embodiment according to the invention, the composition comprises a semiochemical composition, in particular a mixture of feline facial pheromone and cat soothing pheromone, comprising from 2% to 6% by weight of methyl laurate, from 2% to 6% by weight of methyl myristate, from 11% to 15% by weight of methyl palmitate, from 2% to 6% by weight of methyl stearate, from 42% to 50% by weight of methyl oleate, from 4% to 8% by weight of methyl linoleate, from 7% to 13% by weight of dimethyl pimelate and from 6% to 11% by weight of dimethyl azelate; and relates to the non-therapeutic use of said composition for topical, in particular cutaneous, administration to cats, in particular on the skin of a cat, in particular to prevent and manage stress-induced urinary marking behaviour in cats.

In a particular embodiment according to the invention, the composition comprises a semiochemical composition, in particular a dog-soothing pheromone, comprising from 25% to 35% by weight of methyl linoleate, from 25% to 35% by weight of methyl oleate, from 15% to 25% by weight of methyl palmitate, from 10% to 15% by weight of methyl stearate, from 2% to 7% by weight of methyl myristate, from 2% to 6% by weight of methyl pentadecanoate and from 1% to 3% by weight of methyl laurate; and relates to the non-therapeutic use of said composition for topical, in particular cutaneous, administration to dogs, in particular on the skin of a dog, in particular to facilitate adaptation and prevent stress induced by encounters with unknown dogs, humans or animals, and/or to prevent stress reactions induced by external factors such as fireworks, thunderstorms, violent noises or unusual environments.

In a particular embodiment according to the invention, the composition comprises a semiochemical composition, in particular an allomone produced by the uropygial glands of the duck, comprising from 40% to 60% by weight of bis(2-ethylhexyl) adipate and from 40% to 60% by weight of 1-isopropyl-2,2-dimethyltrimethylene diisobutyrate; and relates to the non-therapeutic use of said composition for topical, in particular cutaneous, administration to poultry such as hens, in particular on the skin of a poultry such as a hen, in particular as a repellent allomone, in particular to prevent infestation by parasites such as the poultry red mite (Dermanyssus sp. or Ornithonyssus sylviarum).

Another non-therapeutic use of said composition concerns topical, in particular cutaneous, administration to an animal, for example birds or mammals, in particular on the skin of birds or mammals, or to a human, in particular on human skin, in particular to prevent infestation or stings by invertebrates, in particular haematophagous insects.

In a particular embodiment according to the invention, the composition comprises a semiochemical composition, in particular an equine soothing pheromone, comprising from 35% to 45% by weight of methyl oleate, from 20% to 30% by weight of methyl palmitate, from 20% to 30% by weight of methyl linoleate, from 2% to 7% by weight of dioxolane, from 1% to 5% by weight of methyl laurate and from 1% to 4% by weight of methyl myristate; and relates to the non-therapeutic use of said composition for topical, in particular cutaneous, administration to horses, in particular on the skin of a horse, in particular to prevent social stress in the horse and/or aggressive behaviour induced by encounters with unknown conspecifics, unknown people or animals of other species.

The above-mentioned uses of said composition according to the invention are non-therapeutic uses, and exclude any therapeutic use practised on the animal or human body.

### EXAMPLES

The following examples are given by the way of illustration and therefore should not be construed to limit the scope of the invention.

### Example 1. Oil-In-Water Emulsions (Cream)

Compositions in the form of an oil-in-water emulsion (cream) according to the invention having the composition shown in the following Table 1 was prepared.

TABLE 1

| Compound | Func- | | | Percentage |
|---|---|---|---|---|
| name | tion | INCI | CAS No. | by weight |
| Aqueous | | | | |
| phase | | | | |
| Water | | AQUA | 7732-18-5 | qsp |
| Glycerol | Humec- | GLYCERIN | 56-81-5 | 7.5% |
| | tant | | | |
| Xanthan | Gelling | XANTHAN GUM | 11138-66- | 0.5% |
| gum | agent | | 2 | |
| Oily phase | | | | |
| Montanov L | Emul- | C14-22 ALCOHOLS | | 5.0% |
| | sifying | & C12-20 ALKYL | | |
| | agent | GLUCOSIDE | | |
| Montanov 14 | Emul- | MYRISTYL | 112-72-1 | 2.0% |
| | sifying | ALCOHOL & | | |
| | agent | MYRISTYL | | |
| | | GLUCOSIDE | | |
| Myritol 318 | Emol- | CAPRYLIC/CAPRIC | 73398-61- | 2.0% |
| | lient | TRIGLYCERIDE | 5 | |
| | agent | | | |
| Polysorbate | Emul- | POLYSORBATE 85 | 9005-70-3 | 2.0% |
| 85 | sifying | | | |
| | agent | | | |
| Polysorbate | Emul- | POLYSORBATE 80 | 9005-65-6 | 2.0% |
| 80 | sifying | | | |
| | agent | | | |
| Tinogard TT | Antiox- | PENTAERYTHRITYL | 6683-19-8 | 0.1% |
| | idant | TETRA-DI-T-BUTYL | | |
| | agent | HYDROXYHYDRO- | | |
| | | CINNAMATE | | |
| Aromastat | Antimi- | GLYCERIN/ | 56-81-5 | 1.0% |
| | crobial | PHENETHYL | 60-12-8 | |
| | agent | ALCOHOL | 7377-03-9 | |
| | | CAPRYLHY- | | |
| | | DROXAMIC ACID | | |
| Semio- | | / | / | 2% |
| chemical | | | | |
| composition | | | | |
| (SC1, SC2, | | | | |
| SC3, SC4, | | | | |
| SC5 or SC6) | | | | |
| Perfume | | / | / | 1.0% |
| (optional) | | | | |
| 1% NaOH | | SODIUM | 1310-73-2 | 0.007% |
| solution | | HYDROXIDE | | |
| (only with | | | | |
| SC2 or SC3) | | | | |

### Semiochemical Compositions (SC) Used as an Example in the Formulation of Table 1

SC1: 35% by weight of methyl linoleate, 29% by weight of methyl oleate, 18% by weight of methyl palmitate, 9.18% by weight of 2-(2-butoxyethoxy) ethanol, 4.32% by weight of dimethyl glutarate, 1.62% by weight of dimethyl adipate, 1.62% by weight of dimethyl succinate and 1.26% by weight of dodecane; or
SC2: 11% by weight of methyl stearate, 17% by weight of methyl linoleate, 22% by weight of methyl palmitate, 11% by weight of methyl laurate, 11% by weight of methyl myristate and 28% by weight of methyl oleate; or
SC3: 4.50% by weight of methyl laurate, 4.50% by weight of methyl myristate, 13.40% by weight of methyl palmitate, 4.50% by weight of methyl stearate, 46.60% by weight of methyl oleate, 6.50% by weight of methyl linoleate, 11% by weight of dimethyl pimelate and 9% by weight of dimethyl azelate; or
SC4: 29% by weight of methyl linoleate, 29% by weight of methyl oleate, 18% by weight of methyl palmitate, 13% by weight of methyl stearate, 5% by weight of methyl myristate, 4% by weight of methyl pentadecanoate and 2% by weight of methyl laurate; or
SC5: 50% by weight of bis(2-ethylhexyl) adipate and 50% by weight of 1-isopropyl-2,2-dimethyltrimethylene diisobutyrate;
SC6: from 35% to 45% by weight of methyl oleate, from 20% to 30% by weight of methyl palmitate, from 20% to 30% by weight of methyl linoleate, from 2% to 7% by weight of dioxolane, from 1% to 5% by weight of methyl laurate and from 1% to 4% by weight of methyl myristate.

### Example 2. Process for Preparing Oil-In-Water Emulsions (Creams) According to Example 1

The oil-in-water emulsions (creams) according to Example 1 were prepared according to the following protocol:

### Step 1: Preparing the Paste/Aqueous Phase

1. The glycerin and xanthan gum were weighed and added into a container adapted to contain the final volume of the aqueous phase. The mixture was stirred at room temperature.
2. Once the pasting was complete, all the water was added.
3. After homogenising the aqueous phase (obtaining a homogeneous mixture), the aqueous phase was kept at 70° C. and stirred until step 3.

### Step 2: Preparing the Oily Phase

4. The compounds in the oily phase, namely Montanov L, Montanov 14, Polysorbate 80, Polysorbate 85, Myritol 318, Tinogard TT, Aromastat and the semiochemical composition, were weighed and added into another container adapted to contain the final volume of the oily phase.
5. Once all the compounds were added, the mixture was stirred and heated to 70° C. until a homogeneous mixture was obtained.

### Step 3: Formation of the Oil-In-Water Emulsion

6. When the two phases (aqueous and oily) were perfectly homogeneous, the oily phase was added at 70° C. to the aqueous phase at 70° C. Care was taken to ensure that the emulsion was well agitated throughout this stage. The emulsion was then cooled to room temperature after homogenising the emulsion.
   Step 4: Modification of pH Only with the Above-Mentioned SC2 and SC3 Compositions
7. In the case of compositions comprising the semiochemical composition SC2 or SC3 and in order to increase the pH, a pH adjustment step was carried out by adding a 1% NaOH solution until a pH of between 7 and 8 was obtained.

### Step 5: Perfume

8. Add any perfume with stirring at room temperature.

### Step 6: Recovery of the Oil-In-Water (O/W) Emulsion

The final emulsion obtained was white, opaque and thick.

### Example 3. Organoleptic Measurements of Oil-In-Water Emulsions (Creams) According to Example 1

4 criteria (appearance, colour, odour, texture) were selected for organoleptic measurements.

Appearance: Thick cream

Colour: White

Odour: Characteristic odour

Texture: rich texture (cream).

### Example 4. PH Measurements of Oil-In-Water Emulsions (Creams) According to Example 1

The pH measurements were carried out using a LE 438 IP 67 electrode marketed by Mettler Toledo®. This is a 2-in-1 glass pH electrode with integrated temperature sensor and liquid electrolyte for rapid measurements in the laboratory; for aqueous samples such as acids, bases, etc.

The specifications for this electrode are given in Table 2 below.

TABLE 2

| Measuring range | pH 0-14 | |
|---|---|---|
| Zero point | 7.00 ± 0.25 | |
| Temperature range | 0-80° | C. |
| Temperature sensor | NTC 30 K Ω | |
| Membrane resistance (25° C.) | <250 | MΩ |

| | |
|---|---|
| Reference electrolyte | 3 mol/L KCl solution saturated with AgCl |
| Reference system | Ag/AgCl |
| Reference diaphragm | Gel |

Before any measurements are taken, the pH meter used is calibrated. The nature of the semiochemical composition can affect the pH of the emulsion, as some semiochemicals have an acid pH. For topical application, particularly to the skin, it is therefore necessary to measure the pH of each emulsion obtained and, if necessary, adjust the pH to obtain a pH of between 5 and 8, preferably between 7 and 8.

pH measurements were carried out after each emulsion was obtained and are reported below:
CREAM with SC1: 5.5<pH<7.3
CREAM with SC2: 5.5<pH<6.5
CREAM with SC3: 5.5<pH<6.5
CREAM with SC4: 5.5<pH<7.5
CREAM with SC5: 5.5<pH<7.5
CREAM with SC6: 5.5<pH<7.5

In order to get as close as possible to the pH of animal or human skin, and to avoid causing an irritant skin reaction, the pH was adjusted for the formulations corresponding to CREAM with SC2 and CREAM with SC3.

Following this adjustment, new pH measurements were carried out and are reported below:
CREAM with SC2: 7.0<pH<8.0
CREAM with SC3: 7.0<pH<8.0

For the formulations corresponding to CREAM with SC1, CREAM with SC3 and CREAM with SC4, CREAM with SC6, no pH adjustment step was carried out.

### Example 5. Measuring the Viscosity of Oil-In-Water Emulsions (Creams) According to Example 1

Viscosity is the resistance of a fluid to sliding from one layer to another. The viscosity of each cream must be checked after each production run. Viscosity is influenced by a number of factors, including temperature, the measurement device used, the measurement speed used and the time of measurement during production (measurements at T0 and T+24 h, T+48 h). The viscosity should be measured when the solution is at room temperature, preferably at T+24 h. The viscosity of the oil-in-water emulsions according to example 1 was measured using a Brookfield digital viscometer (model DV-I Prime), mobile S 63 and at a rotation speed of 6 RPM.

The Brookfield DV-I Prime viscometer measures the viscosity of fluids at a given velocity gradient. The principle of operation involves rotating a moving part (which is immersed in the liquid to be tested) by means of a calibrated spring. The measurement range (in centipoises or milliPascal-second) is determined by the speed of rotation of the device, the size and shape of the device, the cross-section where the device is rotating and the full scale of the torque of the calibrated spring.

The specifications of the Brookfield DV-I Prime viscometer are given in Table 3 below.

TABLE 3

| | |
|---|---|
| Speeds (rpm) | 0.0; 0.3; 0.6; 1.5; 3; 6; 12; 30; 60; |
| | 0.5; 1; 2; 2.5; 4; 5; 10; 20; 50; 100 |
| Viscosity range (cP) | 15-2,000,000 |
| Operating medium | Temperature range from 0° C. to |
| | 40° C. (32° F. to 104° F.), 20% to |
| | 80% R.H.: non-condensing |
| Analogue torque output | 0-1 Vdc (torque from 0 to 100%) |
| Accuracy of viscosity measurement | ±1° C.: -of full scale |
| Reproducibility of viscosity | 0.2% of full scale used |
| measurement | |
| Devices used | S61; S62; S63; S64 |

As a result of these measurements, all the oil-in-water emulsions (creams) according to Example 1 had a viscosity >4500 cP or mPa·s.

### Example 6. Comparative Tests of Persistence Studies

The inventors carried out persistence studies on the skin of rabbits by comparing two compositions with two different galenic formulations:
(1) a composition according to the invention in the form of an oil-in-water emulsion (cream) having the composition of Example 1 comprising the semiochemical composition SC1, and
(2) a comparative composition in liquid form comprising the semiochemical composition SC1 diluted in the solvent diethylene glycol monoethyl ether (solvent of petroleum origin).

### Methods Used:

Both studies were carried out using the same protocol:
A sampling medium (compress or cotton bud) was rubbed over the rabbits' skin before applying the compositions to check that compounds naturally present on the rabbits did not interfere with the analyses;
the composition according to the invention and the comparative composition were then applied to the rabbits at the nape of the neck (the area which is the most difficult to access by licking);
each day, a compress or cotton bud was rubbed over the application area on a single rabbit.

The “sampled” rabbit was then removed from the trial to avoid taking two samples from the same rabbit.
The compress or cotton bud was then extracted with a chemical solvent and the extract was analysed by gas chromatography with a mass spectrometry detector to determine whether or not compounds of the semiochemical composition SC1 were present in the rabbit.
For each analysis, so-called “blank” analyses (analysis of solvent alone, extract of compress or cotton bud without composition, extract rubbed onto the rabbits' skin before application of the compositions) were carried out to ensure that the signal observed on the analyses came solely from the product applied to the rabbit.

### Results Obtained:

Study of persistence on rabbit skin using the comparative composition in liquid form (diethylene glycol monoethyl ether)

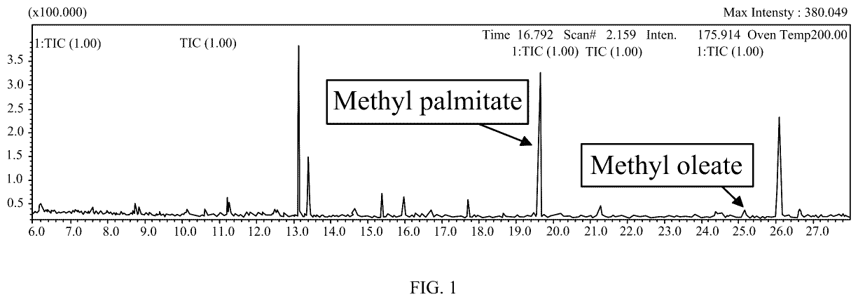

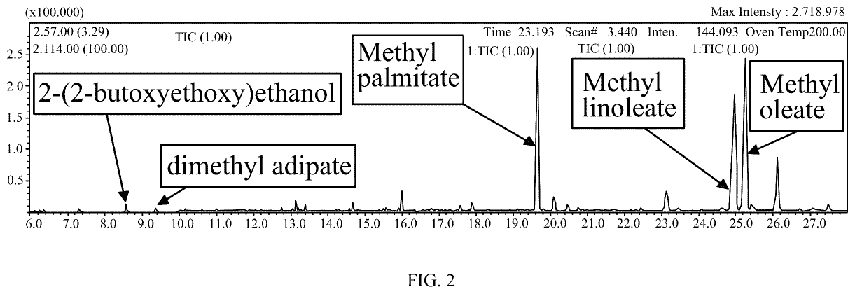

The following active ingredients of semiochemical composition SC1 were identified by gas chromatography on compresses rubbed on the skin of rabbits at the following time intervals:
before submitting the comparative composition (FIG. 1): methyl palmitate and methyl oleate;
24 hours after deposition of the comparative composition (FIG. 2): methyl palmitate, methyl oleate, methyl linoleate, dimethyl adipate, 2-(2-butoxyethoxy) ethanol.

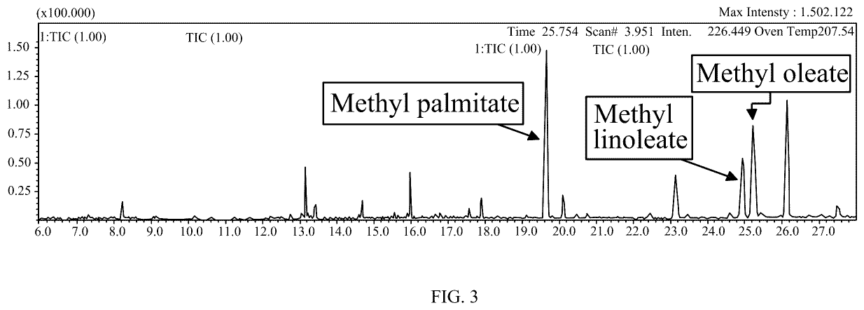

In addition, the quantities of the two active ingredients (methyl palmitate and methyl oleate) already present on the compress before application were lower than those present after application of the comparative composition. Consequently, the SC1 semiochemical composition was considered to be present after 24 hours on the skin of rabbits.
After 2 days (FIG. 3): only the three most abundant active ingredients (methyl linoleate, methyl oleate, methyl palmitate) were still found;
After 3 days: the quantities of active ingredients found were negligible as they were similar to those already present on the compress before the comparative composition was applied.

As a result, the inventors identified that the persistence of the comparative composition in liquid form (diethylene glycol monoethyl ether) lasted 2 days.
Persistence study on the skin of rabbits using the composition according to the invention in the form of an oil-in-water emulsion (cream)

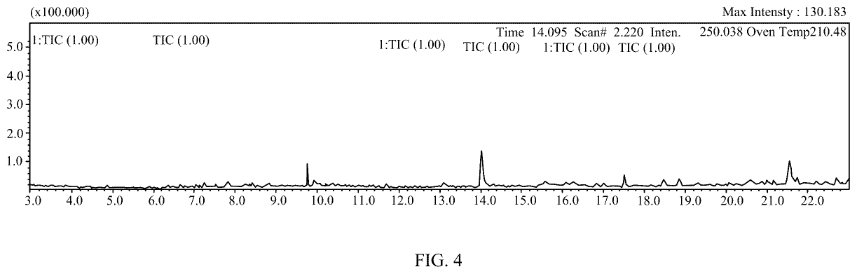

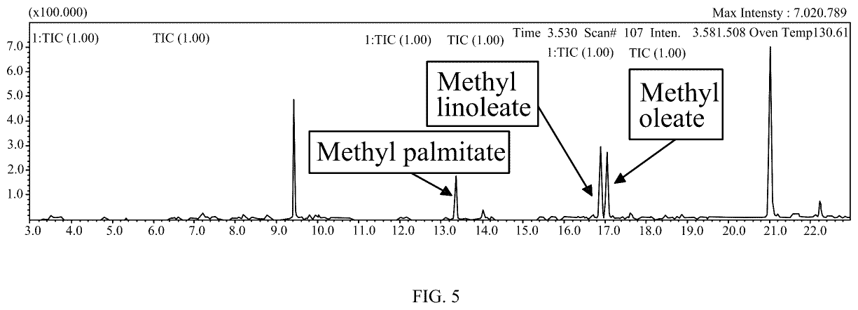

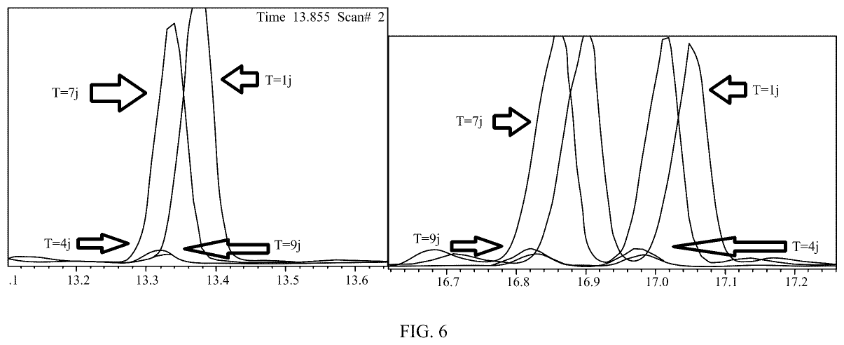

The following active ingredients of semiochemical composition SC1 were identified by gas chromatography on cotton buds rubbed on the skin of rabbits at the following time intervals:
before depositing the composition according to the invention (FIG. 4): no active principle of the semiochemical composition SC1 was found;
24 hours after the deposition of the composition according to the invention (FIG. 5): the most abundant active principles (methyl linoleate, methyl oleate, methyl palmitate) were found;
after 4, 7 and 9 days (FIG. 6): the most abundant active principles (methyl linoleate, methyl oleate, methyl palmitate) were found at varying intensities.

Consequently, the inventors identified that the persistence of the composition according to the invention in the form of an oil-in-water emulsion (cream) having the composition of Example 1 comprising the semiochemical composition SC1 lasted 9 days.

In conclusion, the results of the persistence studies carried out on two compositions with different galenic forms (cream vs liquid) showed that the remanence of the composition according to the invention in the form of an oil-in-water emulsion (cream) having the composition of Example 1 comprising the semiochemical composition SC1 was significantly greater (9 days) than that obtained with the comparative composition in liquid form. The non-therapeutic use of a composition according to the invention in the form of an oil-in-water emulsion (cream) for cutaneous administration to the skin of a rabbit resulted in a prolonged release of the semiochemical composition SC1.

### REFERENCES

Silverstein, R. M., 1977. “Complexity, diversity and specificity of behavior-modifying chemicals; examples from Coleoptera and Hymenoptera,” Chemical Control of Insect Behavior: Theory and Application. Publishers H. H. Shorey & J. J. Mckelvey pp. 231-251 New York: Plenum Press).
Wyatt T. D., 2003 “Pheromones and Animal Behavior. Communication by smell and taste, pp. 1-4 New York: Cambridge University Press.

1. A composition comprising:
a) an aqueous phase comprising water, at least one humectant chosen from polyols and sugars, and at least one gelling agent chosen from polysaccharides,
b) an oily phase comprising at least one emollient agent, at least one emulsifying agent, and a semiochemical composition,
wherein the semiochemical composition comprises semiochemical compounds selected from the group consisting of: (i) a mixture comprising methyl linoleate, methyl oleate and methyl palmitate; and
(ii) a mixture of bis(2-ethylhexyl) adipate and 1-isopropyl-2,2-dimethyltrimethylene diisobutyrate;
wherein the composition is in the form of an oil-in-water (O/W) emulsion and for sustained release of the semiochemical composition.

2. The composition according to claim 1, wherein the semiochemical composition comprises semiochemical compounds selected from the group consisting of:
(a) a mixture of methyl linoleate, methyl oleate, methyl palmitate, 2-(2-butoxyethoxy) ethanol, dimethyl glutarate, dimethyl adipate, dimethyl succinate and dodecane;
(b) a mixture of methyl stearate, methyl linoleate, methyl palmitate, methyl laurate, methyl myristate and methyl oleate;
(c) a mixture of methyl laurate, methyl myristate, methyl palmitate, methyl stearate, methyl oleate, methyl linoleate, dimethyl pimelate and dimethyl azelate;
(d) a mixture of methyl linoleate, methyl oleate, methyl palmitate, methyl stearate, methyl myristate, methyl pentadecanoate and methyl laurate;
(e) a mixture of methyl oleate, methyl palmitate, methyl linoleate, dioxolane, methyl laurate and methyl myristate; and
(f) a mixture of bis(2-ethylhexyl) adipate and 1-isopropyl-2,2-dimethyltrimethylene diisobutyrate.

3. The composition according to claim 1, wherein the semiochemical composition comprises:
(a) from 30% to 40% by weight of methyl linoleate, from 25% to 35% by weight of methyl oleate, from 15% to 25% by weight of methyl palmitate, from 5% to 15% by weight of 2-(2-butoxyethoxy) ethanol, from 2% to 6% by weight of dimethyl glutarate, from 1% to 3% by weight of dimethyl adipate, from 1% to 3% by weight of dimethyl succinate and from 1% to 3% by weight of dodecane; or
(b) from 5% to 15% by weight of methyl stearate, from 10% to 25% by weight of methyl linoleate, from 15% to 25% by weight of methyl palmitate, from 5% to 15% by weight of methyl laurate, from 5% to 15% by weight of methyl myristate and from 25% to 35% by weight of methyl oleate; or
(c) from 2% to 6% by weight of methyl laurate, from 2% to 6% by weight of methyl myristate, from 11% to 15% by weight of methyl palmitate, from 2% to 6% by weight of methyl stearate, from 42% to 50% by weight of methyl oleate, from 4% to 8% by weight of methyl linoleate, from 7% to 13% by weight of dimethyl pimelate and from 6% to 11% by weight of dimethyl azelate; or
(d) from 25% to 35% by weight of methyl linoleate, from 25% to 35% by weight of methyl oleate, from 15% to 25% by weight of methyl palmitate, from 10% to 15% by weight of methyl stearate, from 2% to 7% by weight of methyl myristate, from 2% to 6% by weight of methyl pentadecanoate and from 1% to 3% by weight of methyl laurate; or
(e) from 35% to 45% by weight of methyl oleate, from 20% to 30% by weight of methyl palmitate, from 20% to 30% by weight of methyl linoleate, from 2% to 7% by weight of dioxolane, from 1% to 5% by weight of methyl laurate and from 1% to 4% by weight of methyl myristate; or
(f) from 40% to 60% by weight of bis(2-ethylhexyl) adipate and from 40% to 60% by weight of 1-isopropyl-2,2-dimethyltrimethylene diisobutyrate;
the total sum of the semiochemical compounds present in the semiochemical composition being equal to 100%.

4. The composition according to claim 1, wherein the at least one humectant is chosen from polyols selected from the group consisting of glycerol, propylene glycol, polyethylene glycols (PEGs), xylitol, isomalt, maltitol, mannitol, polydextrose, sorbitol, isomaltitol, lactitol, and mixtures thereof; and/or from sugars selected from the group consisting of fructose, glucose and lactose.

5. The composition according to claim 4, wherein the at least one humectant is glycerol.

6. The composition according to claim 1, wherein the at least one humectant is present in an amount ranging from 4% to 12% by weight, relative to the total weight of the composition.

7. The composition according to claim 1, wherein the polysaccharides are selected from the group consisting of xanthan gum, guar gum, cellulose, cellulose derivatives, maltodextrin, maltodextrin derivatives and mixtures thereof.

8. The composition according to claim 7, wherein the polysaccharides are xanthan gum.

9. The composition according to claim 1, wherein the polysaccharides are present in an amount ranging from 0.05% to 1% by weight, relative to the total weight of the composition.

10. The composition according to claim 1, which comprises from 60% to 95% by weight of water, relative to the total weight of the composition, and/or from 2% to 5% by weight of the semiochemical composition, relative to the total weight of the composition.

11. The composition according to claim 1, wherein the at least one emollient agent is selected from the group consisting of hydrogenated vegetable oils, non-hydrogenated vegetable oils, silicone oils, mineral oils, waxes, fatty alcohols, fatty acids, fatty acid esters, glyceride derivatives, glycerol derivatives.

12. The composition according to claim 11, wherein the at least one emollient agent is a glyceride derivative.

13. The composition according to claim 1, wherein the at least one emollient agent is present in an amount ranging from 0.5% to 8% by weight, relative to the total weight of the composition.

14. The composition according to claim 1, wherein the at least one emulsifying agent is selected from the group consisting of polyoxyethylene sorbitan fatty acid esters, alkyl polyglucosides, and mixtures of at least one alkyl polyglucoside and at least one fatty alcohol.

15. The composition according to claim 1, wherein the at least one emulsifying agent is present in an amount ranging from 2% to 30% by weight, relative to the total weight of the composition.

16. The composition according to claim 1, wherein the oily phase further comprises at least one additive selected from the group consisting of a preservative, a perfume, a masking agent, and mixtures thereof.

17. The composition according to claim 1, which is in the form of a cream.

18. The composition according to claim 1, wherein it comprises:
a) an aqueous phase comprising water, glycerol and xanthan gum,
b) an oily phase comprising at least one C₁₂-C₂₀ alkyl polyglucoside and at least one C₁₄-C₂₂ fatty alcohol, a mixture of myristic alcohol and myristylic glucoside, caprylic/capric acid triglycerides, polyoxyethylene sorbitan fatty acid esters, pentaerythrityl tetra-di-t-butyl hydroxyhydrocinnamate, glycerol/phenyl alcohol/caprylhydroxamic acid, and a semiochemical composition,
the semiochemical composition being present in the composition in the form of an O/W emulsion in an amount ranging from 2% to 5% by weight, relative to the total weight of the composition.

19. A process for preparing the composition in the form of an oil-in-water (O/W) emulsion according to claim 1, comprising the following steps:
a) the at least one gelling agent is pasted into the at least one humectant; then water is added with stirring and at a temperature of between 65° C. and 75° C. so as to obtain a homogeneous aqueous phase;
b) the at least one emollient agent, the at least one emulsifying agent, the semiochemical composition and optionally the at least one additive are mixed, with stirring and at a temperature of between 65° C. and 75° C. so as to obtain a homogeneous oily phase;
c) the oily phase from step b) is added with stirring, to the aqueous phase from step a) so as to obtain an O/W emulsion;
d) optionally, the pH of the emulsion obtained in step c) is adjusted;
e) optionally, a perfume is added;
f) the composition is recovered in the form of an O/W emulsion obtained in step c), d) or e).

20. The process according to claim 19, wherein:
the pasting step a) is carried out at room temperature; and/or
the homogeneous aqueous phase obtained in step a) is kept under stirring at a temperature of between 65° C. and 75° C.; and/or
the homogeneous oily phase obtained in step b) is kept under stirring at a temperature of between 65° C. and 75° C.; and/or
step c) is carried out by mixing the oily phase from step b) in the aqueous phase from step a) at a temperature of between 65° C. and 75° C., and the emulsion is cooled to room temperature; and/or
step d) is carried out by adding a basic solution.

21. Non-therapeutic use of the composition in the form of an oil-in-water (O/W) emulsion according to claim 1, wherein the use comprises topical administration of the composition according to claim 1 to an animal or to a human, for sustained release of the semiochemical composition.
